(19) United States
(12) Patent Application Publication
(10) Pub. No.: US 2009/0275486 A1
(43) Pub. Date: Nov. 5, 2009

(54) NUCLEIC ACID SEPARATION AND PURIFICATION METHOD BASED ON REVERSIBLE CHARGE INTERACTIONS

(76) Inventors: Nurith Kurn, Palo Alto, CA (US); Joe Don Heath, Sunnyvale, CA (US)

Correspondence Address: WILSON, SONSINI, GOODRICH & ROSATI, 650 PAGE MILL ROAD, PALO ALTO, CA, 94304-1050, US

(21) Appl. No.: 12/299,668

(22) PCT Filed: May 16, 2007
(86) PCT No.: PCT/US2007/011856

§ 371 Date: Jun. 30, 2009

Related U.S. Application Data
(60) Provisional application No. 60/801,088, filed on May 16, 2006.

(51) Int. Cl. C40B 50/00; C07H 1/00; C07H 21/04

(52) U.S. Cl. 506/26; 536/25.4; 536/23.1

(57) ABSTRACT

The invention provides a method for purifying nucleic acids using a polycationic reagent and an anionic substrate to form a complex with a nucleic acid to be purified. The complex may be separated from other components of a mixture and the nucleic acid eluted from the complex with a high ionic strength solution or an anionic reagent.

### CROSS-REFERENCE TO RELATED APPLICATIONS

This application claims the benefit of U.S. Provisional Application No. 60/801,088, filed on May 16, 2006, the disclosure of which is incorporated by reference herein in its entirety.

### FIELD OF THE INVENTION

The invention relates to nucleic acid purification, in particular involving a method that includes reversibly binding a nucleic acid to an anionic surface using a polycation to mediate binding between the nucleic acid and the anionic substrate.

### BACKGROUND

Various methods for nucleic acid isolation and purification have developed in recent years. It is desirable to obtain nucleic acids that are substantially free of contaminants which could interfere with analysis or further processing. For example, contaminants include substances that interfere with hybridization or enzyme-catalyzed reactions, substances that degrade nucleic acids, and substances that interfere with detection of a nucleic acid of interest.

Early techniques for isolation of nucleic acids from a complex mixture, such as a cell extract or amplification reaction mixture, included multiple organic extraction and precipitation steps, using hazardous chemicals, such as chloroform and phenol. More recently, methods employing non-sequence specific binding of polynucleotides to solid surfaces have been developed. For example, matrices have been developed involving ion-exchange chromatography (e.g., J. of Chromatography (1990) 508:61-73; Nucleic Acids Research (1993) 21(12):2913-2915; U.S. Pat. Nos. 5,856,192, 5,660,984, and 4,699,717), reverse phase chromatography (e.g., Hirbayashi et al. (1996) J. Chromatography (1996) 722:135-142; U.S. Pat. No. 5,057,426), affinity chromatography (e.g., U.S. Pat. No. 5,712,383), or a combination thereof (e.g., U.S. Pat. No. 5,652,348; J. Chromatography (1983) 270:117-126).

Solid phases developed for use in nucleic acid isolation include silica-based resins. Silica gel particles developed for HPLC are functionalized with anion-exchange moieties which can exchange with nucleic acids under certain salt and pH conditions (e.g., U.S. Pat. Nos. 4,699,717 and 5,057,426). Modern silica-based systems include controlled pore glass, filters embedded with silica particles, silica gel particles, resins containing silica in the form of diatomaceous earth, glass fibers, or mixtures thereof, and are configured to reversibly bind nucleic acids in the presence of chaotropic agents or a high ionic strength buffer. The nucleic acids remain bound to the solid phase during processing steps such as centrifugation or vacuum filtration, and are then eluted by exposing the solid phase to an elution solution such as a low ionic strength buffer.

An advantage of purification methods involving binding of nucleic acid to a solid surface is the ability to wash the bound material using solutions that retain the bound molecules on the solid surface while removing other non-related components, thus resulting in isolation and purification of the polynucleotides of interest from the sample solution. The use of solid surfaces for binding of polynucleotides of interest is desirable as these are easy to manipulate and are amenable for use in routine laboratory procedures, and do not involve the use of hazardous chemicals. Columns containing such solid surfaces are commercially available and are commonly configured as “spin columns” for use with a centrifugation step. Beads are also commercially available, including magnetic beads with a glass/silica based coating, as well as other configurations, including vacuum based devices.

Solid phases in the form of magnetically responsive particles have also been developed for nucleic acid isolation. Such particles bind directly or indirectly to nucleic acids. An example of a system which utilizes direct binding includes magnetically responsive porous glass beads (e.g., U.S. Pat. Nos. 4,233,169; 4,395,271; 4,297,337). However, nucleic acids bind very tightly to glass and may be difficult to remove once bound. Elution efficiency is higher from porous silica based magnetic particles designed to reversibly bind nucleic acids (e.g., MagneSil™ particles from Promega or BioMAG™ particles from PerSeptive Biosystems). Magnetic systems utilizing indirect binding require magnetic particles, an intermediary, and a medium containing nucleic acid to be isolated. A disadvantage with currently available indirect binding magnetic systems is that different solution and/or temperature conditions may be required for intermediary/nucleic acid and intermediary/particle binding reactions, increasing risk of contamination of the isolated nucleic acid end product. An example of an indirect binding system is a streptavidin coated magnetically responsive microsphere with an oligo dT moiety covalently attached to the streptavidin. The streptavidin-oligo dT molecules act as intermediaries for hybridization to the poly A tail of mRNA (e.g., ProActive™ from Bangs Laboratories (Carmel, Ind.) or PolyATract™ from Promega Corp. (Madison, Wis.)).

Most methods developed to date are optimized for purification of high molecular weight polynucleotide, and result in low recovery of low molecular weight polynucleotides. There is a growing need for the isolation and purification of low molecular weight polynucleotides, such small RNA, for example microRNA, fragmented DNA, fragmented and labeled target generated for hybridization to microarrays, and other sequence detection methods.

Methods for non-specific binding of nucleic acid to magnetic particles induced by precipitation using PEG (polyethylene glycol) and other polyalkylene glycols, have also been described (U.S. Pat. No. http://patft1.uspto.gov/netacgi/nph-Parser?Sect1=PTO1&Sect2=HITOFF&d=PALL&p=1&u=%2Fnetahtml%2FPTO%2Fsrchnum.htm&r=1&f=G&1=50&s1=5.898,071.PN.&OS=PN/5,898,071&RS=PN/5,898.071-h0http://patft1.uspto.gov/netacgi/nph-Parser?Sect1=PTO1&Sect2=HITOFF&d=PALL&p=1&u=%2Fnetahtm1%2FPTO%2Fsrchnum.htm&r=1&f=G&1=50&s1=5,898,071.PN.&OS=PN/5,898,071&RS=PN/5,898,071-h25,898,071; Biotechniques (2002) 32:1296-1302; Hawkins, et. al. (1995) Nucleic Acids Res. 23: 4742-4743), and are commercially available. Although effective and amenable for automation, these methods are not suitable for effective purification of small fragments of polynucleotides such as oligonucleotides less than 100 nucleotides in length.

The recent developments in very large scale gene expression analysis using massively parallel analytical tools such as microarray technologies for whole transcriptome or whole genome analysis require the employment of nucleic acid amplification and subsequent target preparation for further analysis. Targets suitable for microarray analysis of whole genomes or whole transcriptome often require fragmentation and labeling of amplification products. Methods for isothermal amplification of either DNA or RNA samples and subsequent fragmentation and labeling of amplification products have been described. Single primer isothermal amplification of DNA and RNA using chimeric DNA/RNA primers has been described (U.S. Pat. Nos. 6,251,639, 6,692,918, and 6,946,251, and U.S. Patent Application No. 2005/0019793). These methods produce single stranded amplification products. Methods for fragmentation and labeling of amplification products which employ the use of non canonical nucleotides have also been described (U.S. Patent Application No. 2004/0005614). Similarly, methods for linear amplification of RNA which employ in-vitro transcription to generate RNA amplification products have also been described (U.S. Pat. Nos. 6,686,156, 5,545,222, and 5,716,785). Purification of fragmented and labeled amplification products for subsequent analysis on microarrays requires the development of suitable methods which will be highly efficient, fast and amenable for automation. Magnetic particle based technologies are commonly used for automated separation of analytes and are commonly employed in automated immunoassays. The attachment of analyte binding entities to magnetic particles is known in the art and is commonly used in laboratory practice (e.g., U.S. Pat. No. 4,935,147). Methods for particle separation which enable the separation of non-magnetic particulate material, by complexation through interaction with polycations has been described (U.S. Pat. No. 4,935,147). The complexation of negatively-charged non-magnetic particles and other particulate materials such as cells or liposomes, with negatively charged magnetic particles through the interaction with polycations, such as polyamines has been demonstrated. The charge based interactions used in these methods are reversible and are not related to the nature of the particulate material to be separated other than the charge distribution. The reversal of complexation can be induced in the presence of polyanions (such as citrate), by changes in the ionic strength of the solution in which the complexes are suspended or by cleavage of the polycation used for the complexation (U.S. Pat. No. 5,405,743).

The major drawback of the various methods developed thus far is their inefficiency with respect to the purification and recovery of low molecular weight polynucleotides as compared with high molecular weight polynucleotides. Most methods developed thus far were optimized for the purification of nucleic acids from biological samples such as genomic DNA and RNA, or purification of amplification products, where amplification products, which are normally shorter than the genomic DNA, are purified and recovered while short primers and probes are preferentially not bound onto the separation matrix. However, there is a need for purification of short oligonucleotides from either biological samples or reaction mixtures, where the short polynucleotides of interest are in a size range up to about 200 nucleotides, using methods which provide high recovery rates, are easy to manipulate and especially suitable for high throughput application and automation. These needs are mostly driven by the fast developments in molecular biology in general and the rapid adoption of genomic technologies in basic research, drug development, and molecular diagnostics.

### BRIEF SUMMARY OF THE INVENTION

The invention provides methods, compositions, and kits for nucleic acid purification.

In one aspect, the invention provides a method for nucleic acid purification, comprising contacting a sample or reaction mixture comprising nucleic acid with a polycationic reagent and an anionic substrate, wherein a nucleic acid-polycation-anionic substrate complex is formed. In one embodiment, the polycationic reagent is polybrene. In one embodiment, the anionic substrate is a carboxylated substrate, such as, for example, carboxylated polystyrene. In one embodiment, the anionic substrate is in the form of microparticles, such as, for example, carboxylated microparticles. In one embodiment, the anionic substrate is in the form of magnetically responsive microparticles, such as, for example, magnetically responsive carboxylated microparticles. The nucleic acid-polycation-anionic substrate complex may be separated from other components of the sample or reaction mixture. In one embodiment, a magnetically responsive anionic substrate is used and separation comprises application of a magnetic field. Nucleic acid may be eluted from the anionic substrate with a high ionic strength solution or an anionic reagent. In one embodiment, citrate is used for elution. In another embodiment, the nucleic acid is eluted in a high ionic strength solution which is suitable for hybridization of the eluted nucleic acid to probes which are immobilized on a solid surface such as a microarray.

In one embodiment, the method for nucleic acid purification comprises separating a nucleic acid-polycation, anionic substrate complex from a sample or reaction mixture, wherein the complex is produced by contacting a sample or reaction mixture comprising nucleic acid with a polycationic reagent and an anionic substrate.

In some embodiments, the nucleic acid to be purified is the product of an amplification reaction and/or a reaction in which a nucleic acid is synthesized from a template, such as, for example, PCR, primer extension, reverse transcription, DNA replication, strand displacement amplification (SDA), multiple displacement amplification (MDA), or template-independent synthesis, such as chemical synthesis or synthesis using a template independent nucleic acid polymerase. In one embodiment, the nucleic acid is the product of a linear isothermal amplification method comprising (a) hybridizing a single stranded DNA template comprising a target sequence with a composite primer, said composite primer comprising an RNA portion and a 3′ DNA portion; (b) extending the composite primer with DNA polymerase; and (c) cleaving the RNA portion of the annealed composite primer with an enzyme that cleaves RNA from an RNA/DNA hybrid such that another composite primer hybridizes to the template and repeats primer extension by strand displacement, whereby multiple copies of the complementary sequence of the target sequence are produced. In one embodiment, the amplification reaction comprises a method comprising (a) extending a first primer hybridized to a target RNA with at least one enzyme comprising RNA-dependent DNA polymerase activity, wherein the first primer is a composite primer comprising an RNA portion and a 3′ DNA portion, whereby a complex comprising a first primer extension product and the target RNA is produced; (b) cleaving RNA in the complex of step (a) with at least one enzyme that cleaves RNA from an RNA/DNA hybrid; (c) extending a second primer hybridized to the first primer extension product with at least one enzyme comprising DNA-dependent DNA polymerase activity and at least one enzyme comprising RNA-dependent DNA polymerase activity, whereby a second primer extension product is produced to form a complex of first and second primer extension products; (d) cleaving RNA from the first primer in the complex of first and second primer extension products with at least one enzyme that cleaves RNA from an RNA/DNA hybrid such that a composite amplification primer hybridizes to the second primer extension product, wherein the composite amplification primer comprises an RNA portion and a 3′ DNA portion; and (e) extending the composite amplification primer hybridized to the second primer extension product with at least one enzyme comprising DNA-dependent DNA polymerase activity; whereby said first primer extension product is displaced, RNA is cleaved from the composite amplification primer and another composite amplification primer hybridizes such that primer extension and strand displacement are repeated, and whereby multiple copies of a polynucleotide sequence complementary to the RNA sequence of interest are generated.

In some embodiments, the nucleic acid to be purified is a nucleic acid fragment, for example, a nucleic acid fragment of about 20 to about 100 nucleotides, about 50 to about 150, about 100 to about 200, about 150 to about 300, or about 250 to a about 500 nucleotides in length. In one embodiment, the fragment is about 10 to about 500 nucleotides in length. In another embodiment, the fragment is about 10 to about 200 nucleotides in length. In some embodiment, a population of nucleic acid fragments is purified, wherein the population comprises fragments of at least about 10 to any of about 20, 50, 100, 150, 200, 250, 300, 350, 400, 450, or 500 nucleotides in length. Often, a population of nucleic fragment of about 100 to about 200 nucleotides in length is purified. In one embodiment, the nucleic acid fragment is a labeled nucleic acid fragment. In one embodiment, the labeled nucleic acid fragment is prepared by a method comprising (a) synthesizing a polynucleotide from a polynucleotide template in the presence of a non-canonical nucleotide, whereby a polynucleotide comprising the non-canonical nucleotide is generated;(b) cleaving a base portion of the non-canonical nucleotide from the synthesized polynucleotide with an enzyme capable of cleaving the base portion of the non-canonical nucleotide, whereby an abasic site is generated; (c) cleaving a phosphodiester backbone of the polynucleotide comprising the abasic site at or near the abasic site; and (d) labeling the polynucleotide at the abasic site; whereby a labeled polynucleotide fragment is generated. In one embodiment, labeling the polynucleotide at the abasic site comprises labeling with terminal deoxynucleotidyl transferase. In one embodiment, labeling the polynucleotide at the abasic site comprises labeling with a label capable of reacting with an aldehyde residue at the abasic site. In one embodiment, trifluoracetic acid salt (ARP) is used for labeling. In one embodiment, the labeled nucleic acid fragment comprises a biotin label. Other methods for fragmentation and/or labeling of the fragmented nucleic acid are known in the art, including methods for labeling during fragmentation, and the resulting nucleic acid fragments may be purified using the methods described herein.

In one embodiment, the nucleic acid to be purified is a fragment of DNA prepared by digestion with an enzyme selected from the group consisting of DNase and a restriction endonuclease. In one embodiment, the nucleic acid to be purified is a fragment prepared by chemical cleavage. In one embodiment, the nucleic acid to be purified is a fragment of RNA prepared by heating the RNA at a temperature suitable to produce RNA fragments.

In another aspect, the invention provides compositions. In some embodiments, the invention provides a composition comprising a reaction mixture for nucleic acid purification according to a method as described herein. In one embodiment, the composition comprises: a polycationic reagent, for example, polybrene; an anionic substrate, for example, a carboxylated substrate, such as a magnetically responsive carboxylated substrate; and a nucleic acid of interest. In one embodiment, the composition comprises a nucleic acid-polycation-anionic substrate complex. In one embodiment, the composition comprises a nucleic acid-polycation-anionic substrate complex and a high ionic strength solution or anionic reagent for elution of the nucleic acid from the complex. In one embodiment, the composition comprises a nucleic acid purified by a method as described herein.

In one aspect, the invention provides kits for nucleic acid purification according to methods as described herein. In some embodiments, kits comprise a polycationic reagent and an anionic substrate, and/or a buffer suitable for formation of a nucleic acid-polycation-anionic substrate complex, and/or a solution suitable for elution of nucleic acid from a nucleic acid-polycation-anionic substrate complex, in packaging. Kits optionally further comprise instructions for use in a method as described herein.

### BRIEF DESCRIPTION OF THE DRAWINGS

FIG. 1. Composition of reaction mixture for enabling binding of nucleic acid (DNA) molecules to negatively charged particles (magnetic particles) A: polycation (for example polybrene); B: Nucleic acid (any size including small fragments and particularly to the examples below, fragmented and labeled single stranded DNA amplification product; C: negatively charge particles (carboxylate magnetic beads).

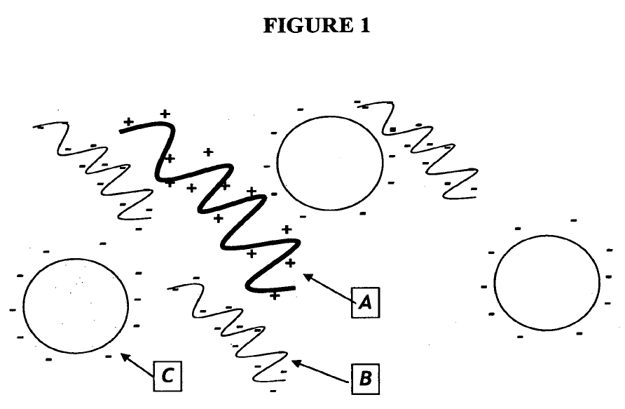

FIG. 2. Charge based binding of nucleic acid molecules to negatively charged particles through interaction with a polycation as binding mediator. The complexes of nucleic acid-polycation-particles can be isolated from the reaction mixture, for example, by magnetic separation (in embodiments in which magnetic particles are utilized), by centrifugation, or by filtration.

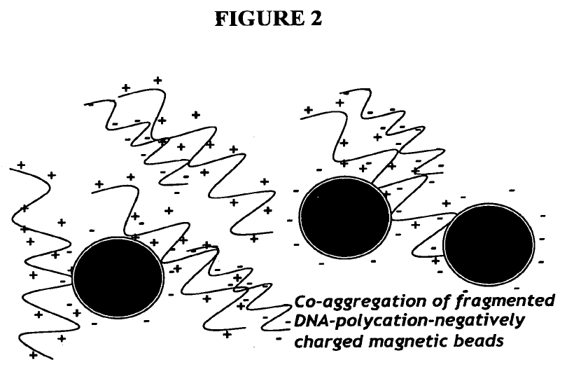

### DETAILED DESCRIPTION OF THE INVENTION

The invention provides methods, compositions, and kits for isolation of nucleic acids from other components of a reaction mixture or sample. In some embodiments, the nucleic acids are oligonucleotide fragments of less than about 200 nucleotides, generally less than about 100 nucleotides in length. Methods of the invention involve formation of a nucleic acid-polycation-anionic solid substrate complex, and separation of the complex from other components in a reaction mixture or sample. In one embodiment, the polycation is polybrene. In one embodiment, the solid substrate is magnetically responsive, and separation includes magnetic separation. Nucleic acids may be bound to the polycation and/or in a nucleic acid-polycation-anionic solid substrate complex in a low ionic strength buffer, and eluted from the complex, after separation of the complex from other components of the reaction mixture or sample, with a high ionic strength solution, or a solution comprising a polyanion such as, for example, citrate. Anions in the elution solution interfere with the charge interaction between the polycation and the nucleic acid. Generally, the cationic charges of the polycation are neutralized by anionic charges in the elution solution. Nucleic acid isolation in accordance with the invention is primarily based on charge interaction between the nucleic acid and polycation, rather than size or sequence of the nucleic acid(s) to be isolated.

### Definitions

“Polynucleotide,” or “nucleic acid,” as used interchangeably herein, refer to polymers of nucleotides of any length, and include DNA, RNA, PNA, or modified forms thereof. The nucleotides can be deoxyribonucleotides, modified nucleotides or bases, and/or their analogs, or any substrate that can be incorporated into a polymer by a polymerase or synthetically. Nucleotides include canonical and non-canonical nucleotides and a polynucleotide can comprise canonical and non-canonical nucleotides. A polynucleotide may comprise modified (altered) nucleotides, such as, for example, modification to the nucleotide structure and or modification to the phosphodiester backbone. Modified nucleotides can be canonical nucleotide or non-canonical (cleavable) nucleotides. A polynucleotide may be further modified after polymerization, such as by conjugation with a labeling component. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, ply-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). It is understood that internucleotide modifications may, e.g., alter the efficiency and/or kinetics of cleavage of the phosphodiester backbone. Further, any of the hydroxyl groups ordinarily present in the sugars may be replaced, for example, by phosphonate groups, phosphate groups, protected by standard protecting groups, or activated to prepare additional linkages to additional nucleotides. The 5′ and 3′ terminal OH can be phosphorylated or substituted with amines or organic capping groups moieties of from 1 to 20 carbon atoms. Other hydroxyls may also be derivatized to standard protecting groups. Polynucleotides can also contain analogous forms of ribose or deoxyribose. sugars that are generally known in the art, including, for example, 2′-O-methyl-, 2′-O-allyl, 2′-fluoro- or 2′-azido-ribose, carboxylic sugar analogs, α-anomeric sugars, epimeric sugars such as arabinose, xyloses or lyxoses, pyranose sugars, furanose sugars, sedoheptuloses, acyclic analogs and abasic nucleoside analogs. One or more phosphodiester linkages may be replaced by alternative linking groups. These alternative linking groups include, but are not limited to, embodiments wherein phosphate is replaced by P(O)S(“thioate”), P(S)S (“dithioate”), “(O)NR₂ (“amidate”), P(O)R, P(O)OR′, CO or CH₂ (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including DNA.

“Oligonucleotide,” as used herein, generally refers to short, generally single stranded, generally synthetic polynucleotides that are generally, but not necessarily, less than about 200 nucleotides in length. The terms “oligonucleotide” and “polynucleotide” and “nucleic acid” are not mutually exclusive. The description above for polynucleotides and nucleic acids is equally and fully applicable to oligonucleotides.

A “primer,” as used herein, refers to a nucleotide sequence (a polynucleotide), generally with a free 3′—OH group, that hybridizes with a template sequence (such as a template RNA, or a primer extension product) and is capable of promoting polymerization of a polynucleotide complementary to the template. A “primer” can be, for example, an oligonucleotide. It can also be, for example, a sequence of the template (such as a primer extension product or a fragment of an RNA template created following RNase cleavage of a template RNA-DNA complex) that is hybridized to a sequence in the template itself (for example, as a hairpin loop), and that is capable of promoting nucleotide polymerization. Thus, a primer can be an exogenous (e.g., added) primer or an endogenous (e.g., template fragment) primer.

A “complex” is an assembly of components. A complex in accordance with the methods described herein may comprise a nucleic acid, a polycationic molecule, and a negatively-charged solid substrate (magnetically charged in methods in which magnetic separation of the complex is used), a nucleic acid and a polycationic molecule, or a polycationic molecule and a negatively-charged solid substrate (magnetically charged in methods in which separation of the complex is used).

A “reaction mixture” is an assemblage of components, which, under suitable conditions, react to form a complex (which may be an intermediate) and/or a product(s). A reaction mixture encompasses any type of suitable components and includes any sample for which separation of nucleic acid is desired, including biological samples.

A “fragment” of a polynucleotide or oligonucleotide is a contiguous sequence of 2 or more bases. In some embodiments, a fragment (also termed “region” or “portion”) is any of about 20, about 25, about 30 about 35 about 40, about 50, about 65, about 75, about 85, about 100, about 125, about 150, about 175, about 200, about 225, about 250, about 300, about 350, about 400, about 450, about 500, about 550, about 600, about 650 or more nucleotides in length. In some embodiments, the fragments can be at least about 20, about 25, about 30 about 35 about 40, about 50, about 65, about 75, about 85, about 100, about 125, about 150, about 175, about 200, about 225, about 250, about 300, about 350, about 400, about 450, about 500, about 550, about 600, about 650 or more nucleotides in length. In other embodiments, the fragments can be less than about 25, about 30 about 35 about 40, about 50, about 65, about 75, about 85, about 100, about 125, about 150, about 175, about 200, about 225, about 250, about 300, about 350, about 400, about 450, about 500, about 550, about 600, about 650 or more nucleotides in length. In some embodiment, these fragment lengths represent an average size in the population of fragments generated using the methods of the invention.

“A”, “an” and “the”, and the like, unless otherwise indicated include plural forms. “A” fragment means one or more fragments.

Conditions that “allow” an event to occur or conditions that are “suitable” for an event to occur, such as polynucleotide binding to a polycationic molecule, and the like, or “suitable” conditions, are conditions that do not prevent such events from occurring. Thus, these conditions permit, enhance, facilitate, and/or are conducive to the event. Such conditions, known in the art and described herein, depend upon, for example, the nature of the polynucleotide sequence, temperature, and buffer conditions. These conditions also depend on what event is desired, such as binding of a polynucleotide to a cationic molecule, formation of a polynucleotide, polycationic molecule, anionic solid substrate complex.

“Microarray” and “array,” as used interchangeably herein, comprise a surface with an array, preferably ordered array, of putative binding (e.g., by hybridization) sites for a biochemical sample (target) which often has undetermined characteristics. In a preferred embodiment, a microarray refers to an assembly of distinct polynucleotide or oligonucleotide probes immobilized at defined positions on a substrate. Arrays are formed on substrates fabricated with materials such as paper, glass, plastic (e.g., polypropylene, nylon, polystyrene), polyacrylamide, nitrocellulose, silicon and other metals, optical fiber or any other suitable solid or semi-solid support, and configured in a planar (e.g., glass plates, silicon chips) or three-dimensional (e.g., pins, fibers, beads, particles, microtiter wells, capillaries) configuration. Probes forming the arrays may be attached to the substrate by any number of ways including (i) in situ synthesis (e.g., high-density oligonucleotide arrays) using photolithographic techniques (see, Fodor et al., Science (1991), 251:767-773; Pease et al., Proc. Natl. Acad. Sci. U.S.A. (1994), 91:5022-5026; Lockhart et al., Nature Biotechnology (1996), 14:1675; U.S. Pat. Nos. 5,578,832; 5,556,752; and 5,510,270); (ii) spotting/printing at medium to low-density (e.g., cDNA probes) on glass, nylon or nitrocellulose (Schena et al, Science (1995), 270:467-470, DeRisi et al, Nature Genetics (1996), 14:457-460; Shalon et al., Genome Res. (1996), 6:639-645; and Schena et al., Proc. Natl. Acad. Sci. U.S.A. (1995), 93:10539-11286); (iii) by masking (Maskos and Southern, Nuc. Acids. Res. (1992), 20:1679-1684) and (iv) by dot-blotting on a nylon or nitrocellulose hybridization membrane (see, e.g., Sambrook et al., Eds., 1989, Molecular Cloning: A Laboratory Manual, 2nd ed., Vol. 1-3, Cold Spring Harbor Laboratory (Cold Spring Harbor, N.Y.)). Probes may also be noncovalently immobilized on the substrate by hybridization to anchors, by means of magnetic beads, or in a fluid phase such as in microtiter wells or capillaries. The probe molecules are generally nucleic acids such as DNA, RNA, PNA, and cDNA but may also include proteins, polypeptides, oligosaccharides, cells, tissues and any permutations thereof which can specifically bind the target molecules.

The term “3′” generally refers to a region or position in a polynucleotide or oligonucleotide 3′ (downstream) from another region or position in the same polynucleotide or oligonucleotide.

The term “5′” generally refers to a region or position in a polynucleotide or oligonucleotide 5′ (upstream) from another region or position in the same polynucleotide or oligonucleotide.

The term “3′-DNA portion,” “3′-DNA region,” “3′-RNA portion,” and “3′-RNA region,” refer to the portion or region of a polynucleotide or oligonucleotide located towards the 3′ end of the polynucleotide or oligonucleotide, and may or may not include the 3′ most nucleotide(s) or moieties attached to the 3′ most nucleotide of the same polynucleotide or oligonucleotide. The 3′ most nucleotide(s) can be preferably from about 1 to about 50, more preferably from about 10 to about 40, even more preferably from about 20 to about 30 nucleotides.

As used herein, “canonical” nucleotide means a nucleotide comprising one the four common nucleic acid bases adenine, cytosine, guanine and thymine that are commonly found in DNA. The term also encompasses the respective deoxyribonucleosides, deoxyribonucleotides or 2′-deoxyribonucleoside-5′-triphosphates that contain one of the four common nucleic acid bases adenine, cytosine, guanine and thymine (though as explained herein, the base can be a modified and/or altered base as discussed, for example, in the definition of polynucleotide). As used herein, the base portions of canonical nucleotides are generally not cleavable under the conditions used in the methods of the invention.

As used herein, “non-canonical nucleotide” (interchangeably called “non-canonical deoxyribonucleoside triphosphate”) refers to a nucleotide comprising a base other than the four canonical bases. The term also encompasses the respective deoxyribonucleosides, deoxyribonucleotides or 2′-deoxyribonucleoside-5′-triphosphates that contain a base other than the four canonical bases. In the context of this invention, nucleotides containing uracil (such as dUTP), or the respective deoxyribonucleosides, deoxyribonucleotides or 2′-deoxyribonucleoside-5′-triphosphates, are a non-canonical nucleotides. As used herein, the base portions of non-canonical nucleotides are capable of being, generally, specifically or selectively cleaved (such that a nucleotide comprising an abasic site is created) under the reaction conditions used in the methods of the invention. Non-canonical nucleotides are generally also capable of being incorporated into a polynucleotide during synthesis of a polynucleotide (during e.g., primer extension and/or replication); capable of being generally, specifically or selectively cleaved by an agent that cleaves a base portion of a nucleotide, such that a polynucleotide comprising an abasic site is generated; comprise a suitable internucleotide connection (when incorporated into a polynucleotide) such that a phosphodiester backbone at an abasic site (i.e., the non-canonical nucleotide following cleavage of a base portion) is capable of being cleaved by an agent capable of such cleavage; capable of being labeled (following generation of an abasic site); and/or capable of immobilization to a surface (following generation of an abasic site), according to the methods described herein. It is understood that the non-canonical nucleotide may, but does not necessarily, require all of the features described above, depending on the particular method of the invention in which the non-canonical nucleotide is to be used. In some embodiments, non-canonical nucleotides are altered and/or modified nucleotides as described herein. Non-canonical nucleotide refers to a nucleotide that is incorporated into a polynucleotide as well as to a single nucleotide.

An “isolated” or “purified” nucleic acid is one that is substantially free of the materials with which it is associated in nature and/or substantially free of the materials with which it is associated in a reaction mixture or sample. “Purifying” a nucleic acid may include separating the nucleic acid from other components of a mixture and/or concentrating the nucleic acid. Generally, the nucleic acid is at least any of about 80, 85, 90, 95, 99, 99.5, or 99.9% pure.

### Methods of the Invention

Methods of the invention provide a fast and effective means for nucleic acid purification, which is based on reversible charge interactions and thus is effective for the isolation and purification of short polynucleotide molecules. Effective purification and isolation of short polynucleotides from solutions, either samples to be analyzed or from reaction mixtures, using currently available methods is ineffective and time consuming. In particular, the use of negatively charged magnetic particles in methods of the invention provides methods suitable for automation as required for large scale genomic analysis.

Methods of the invention may be used to purify DNA and RNA polynucleotides of various lengths, including, but not limited to, small fragments which are generated by laboratory procedures such as amplification, fragmentation and labeling, restriction fragmentation, in vitro transcription, in vitro synthesis, and the like. The methods are particularly useful, but not limited to, the purification of fragmented and labeled targets generated by amplification of DNA or RNA and subsequent analysis, in particular analysis using microarrays. The exemplary use of the method of the invention for the purification steps during the generation of fragmented and biotin labeled targets for gene expression analysis from total RNA isolated from very small biological samples is described in the Examples below. The Ovation™ RNA amplification system is useful for isothermal linear amplification of all transcripts in minute biological samples. The amplification results in the generation of amplified single stranded cDNA which is subsequently fragmented and biotin labeled for gene expression analysis on microarrays, such as the GeneChip™ high density oligonucleotide arrays (Affymetrix). Insofar as labeling of the targets is carried out by a reactive biotin conjugate that is capable of reacting with the surface of the array, it is necessary to purify the short fragmented and labeled targets from the reaction mixture components. The methods of the invention are effective for purification of such fragments and are also advantageously easily adapted for automation, as required for high throughput systems.

Methods of the invention include binding of polynucleotides to a negatively charged solid substrate, such as negatively-charged particles, wherein binding is mediated through charge-based interaction with a polycationic reagent. Various negatively charged particles are known in the art. In one embodiment, beads comprising carboxyl groups are used, for example, carboxylate-derivatized acrylate, latex, or polyacrolein microparticles. (See, e.g., U.S. Pat. Nos. 4,678,814 and 4,935,147.) Beads comprising other negatively charged groups may be used. In some embodiments, negatively-charged magnetically responsive particles are used, which provides advantages for assay procedures due to the ease of automation of particle separation. Such particles are known in the art. (See, e.g., U.S. Pat. Nos. 6,872,578, 6,534,262, and 6,958,372.) Suitable paramagnetic microparticles for use in the instant invention can be obtained, for example, from Bangs Laboratories, Inc., Fishers, Ind. (e.g., Estapor™ carboxylate-modified encapsulated magnetic microspheres) or from Cortex (MagaPhase™ product line).

In methods of the invention, a reaction mixture or sample that contains nucleic acid(s) of interest is contacted with a polycationic reagent a negatively-charged solid matrix to form a nucleic acid-polycation-anionic solid substrate complex. After formation of the complex, the complex is separated from other components of the sample or reaction mixture. Separation may be effected, for example, by washing the substrate to remove unbound materials, by centrifugation, or by filtration. After separation, nucleic acid may be dissociated from the complex by altering conditions to favor dissociation (e.g., by altering pH, temperature, or ionic strength of the buffer) and/or by adding a releasing agent, wherein nucleic acid is released from the complex.

In some embodiments, nucleic acids isolated in accordance with the methods of the invention are fragments, optionally labeled fragments. Polynucleotide fragments may be prepared and/or labeled, for example, as described in U.S. Patent Application No. 2004/0005614. In some embodiments, polynucleotide fragments of any of about 20, about 25, about 30 about 35 about 40, about 50, about 65, about 75, about 85, about 100, about 125, about 150, about 175, about 200, about 225, about 250, about 300, about 350, about 400, about 450, about 500, about 550, about 600, about 650 or more nucleotides in length are isolated. In some embodiments, the fragments can be at least about 20, about 25, about 30 about 35 about 40, about 50, about 65, about 75, about 85, about 100, about 125, about 150, about 175, about 200, about 225, about 250, about 300, about 350, about 400, about 450, about 500, about 550, about 600, about 650 or more nucleotides in length. In other embodiments, the fragments can be less than about 25, about 30 about 35 about 40, about 50, about 65, about 75, about 85, about 100, about 125, about 150, about 175, about 200, about 225, about 250, about 300, about 350, about 400, about 450, about 500, about 550, about 600, about 650 or more nucleotides in length. In some embodiments, the fragments are any of at least about 20, about 25, about 30 about 35 about 40, about 50, about 65, about 75, about 85, about 100, about 125, about 150, about 175, about 200, about 225, about 250, about 300, about 350, about 400, about 450, about 500, about 550, about 600 nucleotides in length, with an upper limit of any of about 25, about 30 about 35 about 40, about 50, about 65, about 75, about 85, about 100, about 125, about 150, about 175, about 200, about 225, about 250, about 300, about 350, about 400, about 450, about 500, about 550, about 600, about 650 nucleotides in length. In some embodiments, these fragment lengths represent an average size in the population of fragments generated using a method for fragmenting nucleic acids.

A “polycationic reagent” or “polycation” as used herein refers to a compound, composition, or material, either inorganic or organic, naturally occurring or synthetic, having at least two positive charged groups. In some embodiments, the polycationic reagent contains at least 10 positively charged groups. In methods of the invention, a polycationic reagent serves as a bridge between a negatively-charged nucleic acid to be purified and a negatively-charged substrate, permitting the nucleic acid to bind indirectly to the substrate. Examples of polycationic reagents include, but are not limited to, polyalkylene amines, such as polyethyleneimine and polypropyleneimine and their lower alky ammonium salts such as Polybrene®, metal ions such as calcium and barium ion, aminodextrans, protamine, positively charged liposomes, and polylysine.

In one embodiment, a cleavable polycation is used. Cleavable polycations are described, for example, in U.S. Pat. No. 5,405,743. The use of a cleavable polycation for mediation of binding of the polynucleotides to negatively charged particles according to the method of the invention permits the dissociation of the isolated and purified polynucleotide from the particles by means of cleaving the polycation and thus does not require the reversal of ionic interaction for elution of the bound polynucleotide.

The negatively-charged substrate to which the polycationic reagent binds may be in the form of particles or beads, either magnetic or non-magnetic. (See, e.g., U.S. Pat. No. 4,935,147.) The particles may be inherently negatively charged or may be treated chemically or physically to introduce a negative charge. Negatively-charged functional groups may be incorporated to render the substrate material anionic, such as carboxylate groups or other anionic groups. In one embodiment, carboxylated polystyrene particles are used.

In some embodiments, magnetic particles are used. The terms “magnetic particles” and “magnetically responsive particles” are used interchangeably herein, and refer to particles that are intrinsically magnetically responsive or have been rendered magnetically responsive, for example, by attachment to a magnetically responsive substance or by incorporation of such a substance into the particles. The magnetic particles can be paramagnetic, ferromagnetic, or superparamagnetic, usually paramagnetic and will have magnetic susceptibilities (X) of at least 5×10⁻⁵ emu/Oecm³, usually at least 4×10⁻⁴ emu/Oecm³. The diameter of the particles should be small, generally in the range from about 5 nm to 1 micron, preferably from about 10 to 250 nm, more preferably from about 20 to 100 nm, most preferably colloidal.

Exemplary of the magnetic component of particles that are intrinsically magnetically responsive are complex salts and oxides, borides, and sulfides of iron, cobalt, nickel and rare earth elements having high magnetic susceptibility, e.g. hematite, ferrite. The magnetic component of other such particles includes pure metals or alloys comprising one or more of these elements.

For the most part the magnetic particles will contain a core of the magnetic component with surface functional groups such as carboxylate groups. Alternatively, the magnetic component can be incorporated into a particle such as, for example, impregnating the substance in a polymeric matrix. However, this procedure frequently yields larger particles. For a more in-depth discussion of the preparation of magnetic particles by this method, see Whitesides, et al. (1983) Trends in Biotechnology 1(5): 144-148 and references cited therein.

Magnetic particles of less than a hundred nanometers in diameter can be made by precipitating iron oxides in the presence or absence of a coating such as a polysaccharide or protein. Magnetic particles of a few microns diameter can also be made by a ball milling process and removing material which is not in the size range of interest. Typically, magnetic particles formed by this latter process are quite polydisperse, and not as generally useful. More useful monodisperse metal oxide suspensions can be prepared by careful control of pH, temperature and concentrations during the precipitation process. Coating the magnetic particles with macromolecules can increase their colloidal stability. This can be done by direct adsorption of high molecular weight polymers or by functionalizing the surface of the particle and then binding macromolecules to the functional groups. Emulsion polymerization and grafting techniques provide a means for coating magnetic particles with polymers.

In methods of the invention using magnetic particles, a reaction mixture or sample containing nucleic acid to be purified is contacted with a polycationic reagent and anionic magnetic particles to form a nucleic acid-polycation-anionic magnetic particle complex. The complexes are separated from other components of the reaction mixture or sample by application of a magnetic current and separation of the magnetic particles from unbound components.

After separation of a complex comprising nucleic acid, polycationic reagent, and anionic substrate, nucleic acid may be eluted from the complex. The elution of the purified polynucleotides from the complex, following binding to the negatively charged particles, separation and washing of the complex, is affected by the choice of ionic strength of the elution solution. High ionic strength solutions, or polyanions, such as citrate, or polymeric anions such as polyacrylate, are effective in dissociation of the complexes. Polyanions suitable for use in elution of nucleic acids from the complexes include, but are not limited to, heparin, dextran sulfate, negatively charged phospholipids vesicles, polycarboxylic acids, such as polyacrylate or polyglutamate. High ionic strength solutions which are suitable for subsequent analysis of the eluted nucleic acids by hybridization can also be used.

In some embodiments, a nucleic acid is eluted from the negatively complex with a releasing agent. As used herein, “releasing agent” refers to a compound, composition, or material, either naturally occurring or synthetic, organic or inorganic, capable of reversing the non-specific binding between negatively-charged molecules, i.e., dissociating such molecules. The releasing agent acts upon the non-specific bond between the molecules. For example, the releasing agent can act to change the pH of the medium to one which is unfavorable or incompatible with the charge interactions between the molecules. The releasing agent can, therefore, be an acid such as a mineral acid or an organic acid or a base such as a mineral base or an organic base. Alternatively, the releasing agent can act to shield ionic interactions and thus can be a high ionic strength solution or a solution of a neutral polymer such as dextran. Alternatively, the releasing agent can have a charge which disrupts the non-specific binding between the oppositely-charged molecules. Exemplary of the latter are be polyelectrolyte salts such as citrate, polyacrylate, dextran sulfate, and the like, as well as other cations, such as, for example, Mg²⁺.

### Compositions

The invention provides compositions comprising reaction mixtures, complexes, and/or end products of the nucleic acid purification procedures described herein.

In some embodiments, the invention provides a reaction mixture for nucleic acid purification, comprising a nucleic acid to be purified, a polycationic reagent, and a negatively charged substrate. In one embodiment, the polycationic reagent is polybrene. In one embodiment, the negatively charged substrate is a carboxylate derivatized substrate, such as a carboxylated polystyrene particle. In one embodiment, the negatively charged substrate is a magnetically responsive particle.

In one embodiment, the invention provides a nucleic acid-polycation-anionic substrate complex. In one embodiment, the invention provides a reaction mixture comprising a nucleic acid-polycation-anionic substrate complex and a high ionic strength solution or a releasing agent.

In one embodiment, the invention provides a nucleic acid purified by a method as described herein.

### Kits

The invention provides kits for carrying out the methods of the invention. Kits comprise components for performing the methods for nucleic acid purification described herein in suitable packaging, optionally further comprising instructions for use in a method as described herein.

The kits of the invention comprise one or more containers comprising any combination of the components described herein, such as one or more polycationic reagents, one or more negatively charged substrates, and/or buffer suitable for binding nucleic acid and polycationic reagent to a negatively charged substrate, and/or elution buffer or releasing agent to elute the nucleic acid from a nucleic acid-polycation-anionic substrate complex.

One or more reagents in the kit can be provided as a dry powder, usually lyophilized, including excipients, which on dissolution will provide for a reagent solution having the appropriate concentrations for performing any of the methods described herein. Each component can be packaged in separate containers or some components can be combined in one container where cross-reactivity and shelf life permit. The kits of the invention may optionally include a set of instructions, generally written instructions, although electronic storage media (e.g., magnetic diskette or optical disk) containing instructions are also acceptable, relating to the use of components of the methods of the invention for the intended nucleic acid purification methods of the invention, and/or, as appropriate, for using the purified nucleic acid products for purposes such as, for example preparing a hybridization probe, expression profiling, preparing a microarray, or characterizing a nucleic acid. The instructions included with the kit generally include information regarding reagents (whether included or not in the kit) necessary for practicing the methods of the invention, instructions on how to use the kit, and/or appropriate reaction conditions.

The component(s) of the kit may be packaged in any convenient, appropriate packaging. The components may be packaged separately, or in one or multiple combinations.

The relative amounts of the various components in the kits can be varied widely to provide for concentrations of the reagents that substantially optimize the reactions that need to occur to practice the methods disclosed herein and/or to further optimize the sensitivity of any assay.

The following examples are intended to illustrate, but not limit, the invention.

### EXAMPLES

### Example 1

### Polybrene and AMPure™ Bead Purification of Fragmentation and Labeling (F&L) Product

### Concept

Polybrene will bind negatively charges molecules (DNA and carboxylated beads) which can then be placed on magnetic stand and washed with H2O. Resuspension of beads in 0.2M citrate should remove the DNA from the beads. The following experiment was performed to test this concept.

### Materials

Polybrene
Unpurified fragmented and biotin labeled cDNA
H2 O
SSC (0.3M citrate)
AMPure™ beads (Agencourt)
1 Dilutions of polybrene in H2O

| | polybrene | H20 |
|---|---|---|
| 1/5 | 10 | 40 |
| 1/10 | 5 | 45 |
| 1/50 | 5 (1/5) | 45 |
| 1/100 | 5 (1/10) | 45 |
| 1/500 | 5 (1/50) | 45 |
| 1/1000 | 5 (1/100) | 45 |

Bead preparation: AMPure beads were washed (1 ml in 1.5 ml tube, spin 16K
2 rpm 1 min, remove supernatant and resuspend in 1 ml H2O.
   Set up binding solutions (add polybrene and EtOH to fragmented and biotin
3. labeled cDNA targets before beads)

| | F&L | | | | polybrene | 100% |
|---|---|---|---|---|---|---|
| tube | cDNA | beads | H2O | polybrene | dilution | EtOH |
| 1 | 50 | 90 | 5 | 0 | | |
| | | (washed) | | | | |
| 2 | 50 | 90 | 0 | 5 | undil | |
| | | (washed) | | | | |
| 3 | 50 | 90 | 0 | 5 | 1/5 | |
| | | (washed) | | | | |
| 4 | 50 | 90 | 0 | 5 | 1/10 | |
| | | (washed) | | | | |
| 5 | 50 | 90 | 0 | 5 | 1/50 | |
| | | (washed) | | | | |
| 6 | 50 | 90 | 0 | 5 | 1/100 | |
| | | (washed) | | | | |
| 7 | 50 | 90 | 0 | 5 | 1/500 | |
| | | (washed) | | | | |
| 8 | 50 | 90 | 0 | 5 | 1/1000 | |
| | | (washed) | | | | |
| 9 | 25 | 90 | 0 | | | 25 |
| 10 | 25 | 90 | 12.5 | | | 12.5 |
| 11 | 25 | 90 | 22.5 | | | 2.5 |
| 12 | 25 | 90 | 25 | | | 0 |

4 Incubate on desktop 5 minutes
5 Place tubes on magnetic rack, wait until solution clears
6 Remove clear solution and save for possible analysis
7 Wash beads on magnetic rack with 70% EtOH twice
8 Remove EtOH and air dry five minutes
9 Resuspend samples 1-8 in 50 μl 20×SSC
10 Resuspend samples 9-12 in 50 μl H2O
11 Measure recovery by Nanodrop

### Results

| Sample ID | ng/μl | 260/280 | Yield (ug) | % recovery |
|---|---|---|---|---|
| 1 | 0.07 | −2.89 | 0.00 | 0.1% |
| 2 | 72.93 | 1.9 | 3.65 | 68.8% |
| 3 | 68.45 | 1.91 | 3.42 | 64.6% |
| 4 | 48.75 | 1.92 | 2.44 | 46.0% |
| 5 | 0.22 | −1.69 | 0.01 | 0.2% |
| 6 | 0.43 | 1.75 | 0.02 | 0.4% |
| 7 | 0.82 | 2.76 | 0.04 | 0.8% |
| 8 | −0.07 | 0.35 | 0.00 | −0.1% |
| 9 | 16.89 | 2.21 | 1.69 | 31.9% |
| 10 | 13.09 | 2.01 | 1.31 | 24.7% |
| 11 | 13.08 | 2.08 | 1.31 | 24.7% |
| 12 | 11.19 | 2.87 | 1.12 | 21.1% |

### Conclusions

Polybrene significantly improved recovery of fragmentation and labeling cDNA using AMPure™ beads.

### Example 2

The experiment described in Example 1 was repeated using the full concentration of polybrene (10 mg/ml in water); wash complex of particles and targets twice with 70% ethanol, and resuspend particles for target elution with IX hybridization buffer, 2× hybridization buffer or 20×SSC. In addition, performance of the method was assessed using washed particles or unwashed particles (Agencourt magnetic particles in binding buffer).

| | Beads | Elution | ng/μl | Yield (ug) | % recovery | to |
|---|---|---|---|---|---|---|
| 2 | washed | 1X hybe | 70.26 | 3.51 | 66.3% | 21 |
| 6 | unwashed | 1X hybe | 25.83 | 1.29 | 24.4% | |
| 3 | washed | 2X hybe | 69.57 | 3.48 | 65.6% | |
| 7 | unwashed | 2X hybe | 20.26 | 1.01 | 19.1% | |
| 4 | washed | 20X SSC | 59.23 | 2.96 | 55.9% | |
| 8 | unwashed | 20X SSC | 23.09 | 1.15 | 21.8% | |
| | DyeEx1 | | 97.74 | 4.40 | 83.0% | |
| | DyeEx2 | | 96.77 | 4.35 | 82.2% | |

### Conclusions

AMPure beads should be removed from binding buffer and resuspended in water.

1× hybridization buffer is sufficient to elute target

### Comparison of Method of the Invention to Spin Column Purification Procedure for Binding to GeneChip™ Array

GeneChip™ arrays (U133A v2) data summary:

| | Scale | | |
|---|---|---|---|
| | Factor | Background | % P |
| DyeEx purified | 0.991 | Avg: 44.48 | 71.00% |
| target | | | |
| Polybrene | 0.798 | Avg: 46.52 | 72.00% |
| magnetic particles | | | |
| based target | | | |
| purification | | | |

Parameters that are important for performance with respect to gene expression analysis on an array using fragmented and biotin labeled targets include the effective recovery yield of the purified fragmented and labeled targets and the removal of the free reactive biotin conjugate. These performance parameters will affect GeneChip™ array results as reflected by background signals (background due to the reaction of the reactive biotin conjugate with the array surface) and the percent of transcripts detected by hybridization to the high density oligonucleotide array (GeneChip™ arrays, Affymetrix), as denoted by % P. The results of comparison of the performance of targets prepared by the Ovation™ system and purified using either size exclusion spin column, DyeEx, or the method of the invention, demonstrate equal of better performance of the target purified by the method of the invention. The method is easy to perform, highly reproducible and suitable for automation.

### Example 3

### Purification of Fragmented and Biotin Labeled Targets Generated by RNA Amplification using the Ovation™ System with Polybrene and AMPure™ Magnetic Beads

### Concept

Attempt to increase recovery of fragmented and labeled cDNA by increasing the amount of polybrene, increase beads, increase volume of washed beads thereby decreasing the ionic strength of the cDNA-polybrene mix. The following experiment was performed to test this concept.

### Materials

Polybrene
(10 ug/ml)
unpurified F&L
cDNA
1× hybe cocktail
AMPure beads
1 Make 1× washed beads
   spin down 0.5 mL beads at 16K for 1′
   Remove sup
   Resuspend in 0.5 ml H2O
2 Make 2× washed beads
   spin down 0.5 mL beads at 16K for 1′
   Remove sup
   Resuspend in 0.25 ml H2O
3 Set up purifications
   Mix cDNA and polybrene by pipette
   a mixing
   b Add beads and mix by pipette mixing
   c Incubate at RT for 5 minutes
   d Place on mag stand
   e After 5-10 minutes or clear, remove sup
   f wash beads 200 μl 70% EtOH, sit on mag stand 2 minute, remove EtOH
   g repeat
   remove all residual EtOH reasonably possible
   i add 50 μl 1× hybe cocktail. Resuspend. Sit 5 minutes RT.
   j Place on mag stand 5-10 minutes until clear
   k remove sup and measure cDNA on Nanodrop

| | F&L | | | | | | | |
|---|---|---|---|---|---|---|---|---|
| | cDNA | polybrene | 1X beads | 2X beads | | | Effective | % |
| well # | μl | μl | μl | μl | ng/μl | 260/280 | yield | recovery |
| 1 | 25 | 2.5 | 45 | | 35.39 | 1.84 | 3.539 | 66.8% |
| 2 | 25 | 2.5 | 75 | | 41.35 | 1.84 | 4.135 | 78.0% |
| 3 | 25 | 2.5 | | 22.5 | 41.02 | 1.87 | 4.102 | 77.4% |
| 4 | 25 | 2.5 | | 45 | 43.32 | 1.84 | 4.332 | 81.7% |
| 5 | 25 | 5 | 45 | | 38.73 | 1.89 | 3.873 | 73.1% |
| 6 | 25 | 5 | 75 | | 37.74 | 1.88 | 3.774 | 71.2% |
| 7 | 25 | 5 | | 22.5 | 37.52 | 1.91 | 3.752 | 70.8% |
| 8 | 25 | 5 | | 45 | 37.88 | 1.83 | 3.788 | 71.5% |

### Example 4

### Protocol for Polybrene-Aided Bead Cleanup of Ovation™ Biotin cDNA and Fragmented and Labeled cDNA Target (Ovation™ RNA Amplification and Biotin Labeling System Product)

The protocol is designed for purification of amplified cDNA, prior to fragmentation and labeling, using AMPUre™ magnetic particles, according to Agencourt's protocol, elution of purified cDNA in fragmentation reaction buffer, performing fragmentation and labeling according to the Ovation™ protocol, in the presence of the magnetic beads, and purifying the fragmented and labeled target according to the method described herein, for further analysis of gene expression by hybridization to GeneChip™ high density arrays.

### Materials

Ovation Biotin amplified cDNA (unfragmented and unpurified)
Polybrene (10 μg/μl) (cat# TR-1003-G, Specialty Media 1-888-209-8870)
Agencourt AMPure beads
H2O
Fresh 70% EtOH
SPRIPlate 96R magnet (Agencourt)
1× GeneChip Hybridization Buffer

### Procedure

1. Swirl or vigorously shake AMPure bottle to mix beads thoroughly.

2. Add 144 μl of resuspended AMPure beads to each 80 μl SPIA reaction

3. Mix the samples completely by pipetting up and down.

4. Let sit at RT for 5 minutes.

5. Place one of the pair of strip tubes or PCR plates on magnetic plate.

6. Wait 10 minutes or until solution is clear (beads will form a donut ring).

7. Carefully remove and discard clear supernatant without disturbing donut ring of beads.

8. Transfer bead-sample mix from second plate into correct wells/tubes of 1^{st} plate.

9. Wait 10 minutes or until solution is clear (beads will form a donut ring).

10. Carefully remove and discard clear supernatant without disturbing donut ring of beads.

11. Leaving the tubes or plate on the SPRIPlate 96R, added 200 μl 70% EtOH.

12. Allow solution to clear before carefully removing and discarding EtOH without disturbing donut ring of beads.

13. Repeat steps 11-12.

14. Allow to air dry no more than 5 minutes.

15. Remove strip tubes or PCR plate from magnetic rack.

16. Add 35 μl IX Fragmentation Master Mix 1 using components from Ovation Biotin kit (25 μl H2O, 5 μl F1, 5 μl F2) to each tube.

17. Pipette up and down to resuspend beads.

18. Close strip tube caps or seal PCR plate.

19. Incubate strip tubes or PCR plate in thermal cycler at 50 degrees for 30 minutes.

20. Remove from thermal cycler.

21. Add 5 μl F3 and 2.5 μl F4 to each well. Mix thoroughly by pipetting.

22. Reseal and incubate in thermal cycler at 50 degrees for 30 minutes.

23. Remove from thermal cycler.

24. Add 5 μl of 10 mg/ml polybrene to each well and mix completely by pipetting.

25. Let sit at RT for 5 minutes.

26. Place strip tube or PCR plate on magnetic plate.

27. Wait 5 minutes or until solution is clear (beads will form a donut ring).

28. Carefully remove and discard clear supernatant without disturbing donut ring of beads.

29. Add 200 μl 70% EtOH to each well. Wait 1 minute, leaving strip tube or PCR plate on magnetic plate.

30. Carefully remove EtOH without disturbing donut ring of beads.

31. Repeat steps 8-9.

32. If necessary, remove residual EtOH from bottom of tube with small multichannel pipette.

33. Let air dry for no more than 5 minutes.

34. Remove strip tubes or PCR plate from magnetic rack.

35. Add 100 μl 1× GeneChip Hybridization Buffer to each well.

36. Pipette mix or seal/cap and gently vortex to return beads to solution.

37. Allow to sit at RT for 5 minutes.

38. Place strip tubes or plate on magnetic rack.

39. Wait 5 minutes or until solution is clear (beads will form a donut ring).

40. Carefully transfer clear supernatant to a fresh container (tube or PCR plate) without disturbing donut ring of beads.

41. Measure concentration of fragmented cDNA in supernatant on Nanodrop using 1× GeneChip Hybridization Buffer as blank.

All publications, patents, and patent applications cited herein are hereby incorporated by reference in their entireties for all purposes and to the same extent as if each individual publication, patent, or patent application were specifically and individually indicated to be so incorporated by reference.

Although the foregoing invention has been described in some detail by way of illustration and examples for purposes of clarity of understanding, it will be apparent to those skilled in the art that certain changes and modifications may be practiced without departing from the spirit and scope of the invention. Therefore, the description should not be construed as limiting the scope of the invention, which is delineated by the appended claims.

We claim:

1. A method for purifying a nucleic acid, comprising:
contacting a sample or reaction mixture comprising said nucleic acid with a polycationic reagent and an anionic substrate, wherein a nucleic acid-polycation-anionic substrate complex is formed;
separating said nucleic acid-polycation-anionic substrate complex from other components of said sample or reaction mixture; and
eluting said nucleic acid from said nucleic acid-polycation-anionic substrate complex.

2. A method according to claim 1, wherein said polycationic reagent is polybrene.

3. A method according to claim 1, wherein said anionic substrate is magnetically responsive, and said separating comprises application of a magnetic field.

4. A method according to claim 1, wherein said anionic substrate is a carboxylated substrate.

5. A method according to claim 4, wherein said carboxylated substrate is carboxylated polystyrene.

6. A method according to claim 1, wherein said nucleic acid is eluted from said anionic substrate with a high ionic strength solution.

7. A method according to claim 6, wherein said high ionic strength solution is suitable for hybridization of the eluted nucleic acid to probes.

8. A method according to claim 7, wherein said probes are immobilized on a substrate.

9. A method according to claim 8, wherein said probes are in the form of a microarray.

10. A method according to claim 1, wherein said nucleic acid is eluted from said anionic substrate with an anionic reagent.

11. A method according to claim 10, wherein said anionic reagent is citrate.

12. A method according to claim 1, wherein said nucleic acid is produced in an amplification reaction.

13. A method according to claim 1, wherein said nucleic acid is synthesized from a nucleic acid template comprising DNA or RNA.

14. A method according to claim 1, wherein said nucleic acid is produced by a method selected from the group consisting of polymerase chain reaction (PCR), primer extension, reverse transcription, DNA replication, strand displacement amplification (SDA), multiple displacement amplification (MDA), and template-independent synthesis.

15. A method according to claim 1, wherein said nucleic acid is produced by a linear isothermal amplification method comprising:
(a) hybridizing a DNA template comprising a target sequence with a composite primer, said composite primer comprising an RNA portion and a 3′ DNA portion;
(b) extending the composite primer with DNA polymerase;
(c) cleaving the RNA portion of the annealed composite primer with an enzyme that cleaves RNA from an RNA/DNA hybrid such that another composite primer hybridizes to the template and repeats primer extension and strand displacement, whereby multiple copies of the target sequence are produced.

16. A method according to claim 1, wherein said nucleic acid is produced by a linear isothermal amplification method comprising:
(a) extending a first primer hybridized to a target RNA with at least one enzyme comprising RNA-dependent DNA polymerase activity, wherein the first primer is a composite primer comprising an RNA portion and a 3′ DNA portion, whereby a complex comprising a first primer extension product and the target RNA is produced;
(b) cleaving RNA in the complex of step (a) with at least one enzyme that cleaves RNA from an RNA/DNA hybrid;
(c) extending a second primer hybridized to the first primer extension product with at least one enzyme comprising DNA-dependent DNA polymerase activity and at least one enzyme comprising RNA-dependent DNA polymerase activity, whereby a second primer extension product is produced to form a complex of first and second primer extension products;
(d) cleaving RNA from the first primer in the complex of first and second primer extension products with at least one enzyme that cleaves RNA from an RNA/DNA hybrid such that a composite amplification primer hybridizes to the second primer extension product, wherein the composite amplification primer comprises an RNA portion and a 3′ DNA portion; and
(e) extending the composite amplification primer hybridized to the second primer extension product with at least one enzyme comprising DNA-dependent DNA polymerase activity; whereby said first primer extension product is displaced, RNA is cleaved from the composite amplification primer and another composite amplification primer hybridizes such that primer extension and strand displacement are repeated, and whereby multiple copies of a polynucleotide sequence complementary to the RNA sequence of interest are generated.

17. A method according to claim 1, wherein said nucleic acid is a fragment of about 10 to about 500 nucleotides in length.

18. A method according to claim 27, wherein said fragment is about 10 to about 200 nucleotides in length.

19. A method according to claim 17, wherein said fragment is prepared by a method comprising:
(a) synthesizing a polynucleotide from a polynucleotide template in the presence of a non-canonical nucleotide, whereby a polynucleotide comprising the non-canonical nucleotide is generated;
(b) cleaving a base portion of the non-canonical nucleotide from the synthesized polynucleotide with an enzyme capable of cleaving the base portion of the non-canonical nucleotide, whereby an abasic site is generated;
(c) cleaving the phosphodiester backbone of the polynucleotide comprising the abasic site at or near the abasic site, whereby a polynucleotide fragment is generated.

20. A method according to claim 19, further comprising:
(d) labeling the polynucleotide at the abasic site; whereby a labeled polynucleotide fragment is generated.

21. A method according to claim 20, wherein labeling the polynucleotide at the abasic site comprises labeling with terminal deoxynucleotidyl transferase.

22. A method according to claim 20, wherein the labeled polynucleotide fragment comprises a biotin label.

23. A nucleic acid purified according to the method of claim 1.

24. A method for purifying a nucleic acid, comprising:
separating a nucleic acid-polycation, anionic substrate complex from other components of a sample or reaction mixture, wherein said complex is produced by contacting a sample or reaction mixture comprising said nucleic acid with a polycationic reagent and an poly-anionic substrate; and
eluting said nucleic acid from said nucleic acid-polycation-anionic substrate complex.

25. A kit for nucleic acid purification, comprising a polycationic reagent, an anionic substrate, and a buffer suitable for formation of a nucleic acid-polycation-anionic substrate complex.

26. A kit according to claim 25, wherein the polycationic reagent is polybrene and the anionic substrate is selected from the group consisting of carboxylated polystyrene, carboxylated beads, and carboxylated magnetic particles.

27. A kit according to claim 26, further comprising an elution reagent selected from a high ionic strength solution and an anionic reagent.

28. A kit according to claim 26, comprising instructions for use in a method for nucleic acid purification comprising:
contacting a sample or reaction mixture comprising said nucleic acid with a polycationic reagent and an anionic substrate, wherein a nucleic acid-polycation-anionic substrate complex is formed;
separating said nucleic acid-polycation-anionic substrate complex from other components of said sample or reaction mixture; and
eluting said nucleic acid from said nucleic acid-polycation-anionic substrate complex.

29. A method according to claim 1, wherein said nucleic acid is selected from the group consisting of mRNA, cDNA, and genomic DNA, synthetic RNA, and synthetic DNA.

30. A method according to claim 20, wherein (d) comprises labeling the polynucleotide fragment at the abasic site with a label capable of reacting with an aldehyde residue at the abasic site.

31. A method according to claim 20, wherein the abasic site is labeled with trifluoroacetic acid salt (ARP).

32. A method according to claim 17, wherein said nucleic acid is DNA and said nucleic acid fragment is prepared by digestion with an enzyme selected from the group consisting of DNase and a restriction endonuclease.

33. A method according to claim 17, wherein said nucleic acid fragment is prepared by chemical cleavage.

34. A method according to claim 17, wherein said nucleic acid is RNA and said nucleic acid fragment is prepared by heating the RNA at a temperature suitable to produce fragments.
